(19) United States
(12) Patent Application Publication
(10) Pub. No.: US 2025/0367337 A1
(43) Pub. Date: Dec. 4, 2025

(54) OZONE SPRAY METHODS

(71) Applicant: SEEGROW SOLUTIONS LIMITED, Hampshire (GB)

(72) Inventor: Stephen JR NOEL, Old Arlesford, Hampshire (GB)

(21) Appl. No.: 18/709,363

(22) PCT Filed: Nov. 11, 2022
(86) PCT No.: PCT/GB2022/052869

§ 371 Date: May 10, 2024

(30) Foreign Application Priority Data
Nov. 11, 2021 (GB) 2116273.0

(51) Int. Cl. A61L 2/20; A61L 2/18

(52) U.S. Cl. CPC A61L 2/202; A61L 2/18; A61L 2/183

(57) ABSTRACT

The invention relates to methods of controlling one or more pathogens comprising spraying ozone and one or more pathogen reducing compound(s), such as flavonoids and/or nano-coatings, onto grassed, artificial or hybrid playing surfaces.

### FIELD OF THE INVENTION

The invention relates to methods of controlling one or more pathogen comprising spraying ozone and one or more pathogen reducing compound(s), such as flavonoids and/or nano-coatings, onto grassed, artificial or hybrid playing surfaces.

### BACKGROUND TO THE INVENTION

Sports playing surfaces such as artificial, natural grassed or hybrid playing surfaces may suffer from infestations of pathogens. Agricultural crops, fields or machinery may also suffer from soil pests leading to plant disease. For example, parasitic nematodes such as root knot nematodes (Meloidogyne) are sedentary parasites and may establish long-term infections within roots that are often damaging to commercial turf grasses or crops such as potato.

Artificial pitches may also suffer from infestation of pathogens such as bacteria, virus or the ike.

The turf grass industry is a multi-billion pound a year business and is one of the fastest growing segments of horticulture. Preventing turf grass diseases is vital in providing a high-quality performance of the playing surface. Millions are spent on fungicides and other pathogen control methods to implement and manage disease control.

Crop damage by pathogens such as nematodes is c$174bn cost in world-wide in agriculture. Synthetic pesticides and other chemicals may be used to combat soil pests or other pathogens. However, such chemicals may be toxic and cause substantial environmental damage. Increasingly, the use of such chemicals is restricted in the amounts and locations where they can be used.

In view of such issues, natural nematicides derived from garlic have been developed. Another common natural nematicide is obtained from neem cake, the residue obtained after cold pressing the fruit and kernels of the neem tree. Soil steaming can also be used to kill pathogens such as nematodes. However, little success has been achieved in finding safe effective replacements for the toxic but efficacious convention pesticides. Consequently, there remains a need to develop environmentally safe, efficacious methods of controlling pathogens such as nematodes.

There remains a need to develop methods of controlling pathogens whilst maintaining beneficial bacterial and/or fungi in the soil of grassed or hybrid pitch surfaces.

There remains a need to develop methods of controlling pathogens on artificial pitch surfaces for sustained periods of time.

It is an aim of certain embodiments of the present invention to at least partially mitigate the problems associated with the prior art.

It is an aim of certain embodiments of the present invention to provide improved compositions for controlling pathogens and improved methods for delivering such compositions to a site of infection.

### Summary of Certain Embodiments of the Invention

The invention relates to compositions and methods for controlling pathogens. The compositions of the invention may be delivered by spraying onto the surface of sports pitches, playing surfaces or the like. In addition, the compositions of the invention may be used to combat infections of any ground care machinery or artificial sports playing surfaces with pathogens.

Ozone is highly reactive with many organic compounds. The effectiveness of ozone (aqueous and gaseous) has been developed as an alternative sanitizing technology to common conventional disinfectants in reducing the microbial contamination of water and/or air.

However, ozone is challenging to use in outdoor field settings as it degrades quickly after production. In addition, delivering ozone to where its effectiveness can be maximised is difficult.

The invention relates, in part, to the development of methods of controlling pathogens by delivering ozone to the sites of infection of grassed, artificial or hybrid pitches that may be used for sport, leisure, or the like.

The invention also incorporates the manufacture of ozonated water which is used as a carrier. The ozone may then be combined with one or more additional pathogen reducing compounds. Advantageously, combining ozone with pathogen reducing compounds such as flavonoids significantly impacts pathogens such as parasitic nematodes without adversely affecting beneficial fungi or other microbes in the soil. Unexpectedly, the use of flavonoids also increases the duration in which the ozone is effective on the surface types described herein and enhances the recovery of the turf grass or other agricultural crops in the soil following the ozone treatment.

The compositions and methods of the invention also relate to the application of nano-coatings to the pitch surface. For example, the pathogen reducing compounds may provide nanoparticles having hydrophilic and/or photocatalytic properties to the surface of the pitch. For example, pathogen reducing compounds comprising metal oxides (e.g., titanium dioxide or the like) may be sprayed onto a surface during or after treatment with the ozone. Without being bound by theory, such nano-coatings may provide prolonged protection from recontamination by volatile organic compounds. Unexpectedly, the application of the combination of ozone with the nano-coating (e.g., onto artificial pitch surfaces) provides significantly increased protection from recontamination (e.g., six months or more) as compared to treatment with ozone or the nano-coating alone.

In certain embodiments, the ozone and/or additional pathogen reducing compounds are combined with one or more additional oxidizing reagents such as hydrogen peroxide and/or oxygen (O₂). Advantageously, the use of such additional oxidizing reagents can enhance the activity of the ozone.

In certain embodiments, the ozone is combined with an acid. Advantageously, the use of acids such as citric acid or CO₂ to lower pH may act to maintain ozone in an active state in water for a longer period of time.

The compositions and methods of the invention are environmentally friendly as compared to traditional chemical treatments. In addition, they are cost-effective and allow treatment of diseases for which there may be no (or only limited) available control agents.

Accordingly, the invention provides a composition for controlling pathogens, wherein the composition comprises ozone and one or more pathogen reducing compounds (e.g., flavonoids and/or nano-coatings). The ozone (either in liquid or gaseous form) may also be used with one or more additional oxidizing reagents such as oxygen and/or hydrogen peroxide.

The invention further provides methods of controlling one or more pathogens comprising delivering, by spraying, an effective amount of ozone onto the surface of a grassed, artificial or hybrid pitch.

The invention also provides methods of controlling one or more pathogens comprising delivering, by spraying, an effective amount of ozone and pathogen reducing compounds (e.g., flavonoids and/or nano-coatings) to sites of infection, including, for example, agricultural crops or associated agricultural or other machinery.

For example, the ozone and/or pathogen reducing compounds (e.g., flavonoids and/or nano-coatings) may be applied in an amount effect to maintain a ratio of fungi to bacteria of about 0.5 to about 1.5 (e.g., about 1:about 1). Such ratios are particularly effective for nutrient cycling in turf grass.

In preferred embodiments, a spray nozzle is used to deliver ozonated water, at a strength between about 0.001 ppm to about 50 ppm, onto the surface of artificial, hybrid and/or natural grass surfaces for the purposes of controlling pathogens.

In such embodiments, the spray nozzle may be configured to mix the ozonated water (or non-ozonated water delivered from the same holding tank) with the pathogen-reducing compound(s) by combining flow from at least two separate streams. Advantageously, this prevents contamination of the ozonated water with the pathogen-reducing compound(s) as they are kept separate up to the point of delivery in the spray.

In one embodiment, ozonated water and flavonoid(s) are mixed using a spray nozzle configured to combine flow from at least two separate streams. In such embodiments, the ozonated water and flavonoids may be delivered at the same time.

Alternatively, non-ozonated water and flavonoid(s) may be mixed using a spray nozzle configured to combine flow from at least two separate streams. In such embodiments, the ozonated water and flavonoids may be delivered at different times. For example, the flavonoids (mixed with non-ozonated water) may be delivered before or after treatment with the ozonated water. Advantageously, these different configurations may be applied using the same spray system.

In another embodiment, nano-coatings are applied to the pitch surface during and/or after treatment with the ozone. For example, an effective amount of ozonated water may be sprayed onto an artificial pitch surface. Following this treatment, non-ozonated water (e.g., from the same holding tank as the ozonated water) and the nano-coating may be mixed using a spray nozzle configured to combine flow from at least two separate streams. Advantageously, this may provide prolonged protection from recontamination by volatile organic compounds.

In some embodiments, a retractable spray lance or the like may be used. Advantageously, this allows treatment of hard-to-reach areas and equipment wash down.

Embodiments of the present invention will now be described hereinafter, by way of example only, with reference to the accompanying drawings in which:

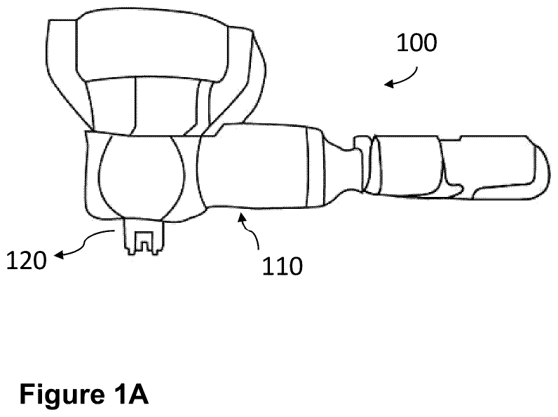

FIG. 1A illustrates a modified spray valve used in the methods of the invention (100). An air measurement cap (110) is configured to draw a second stream of gas or liquid (e.g., comprising the pathogen reducing compound(s)) into a first stream (e.g., ozonated or non-ozonated water) being delivered to a spray nozzle (120). FIG. 1B shows the parts which make up the cap (110) and spray nozzle (120) including O-rings (130, 140) and adaptors (150, 160).

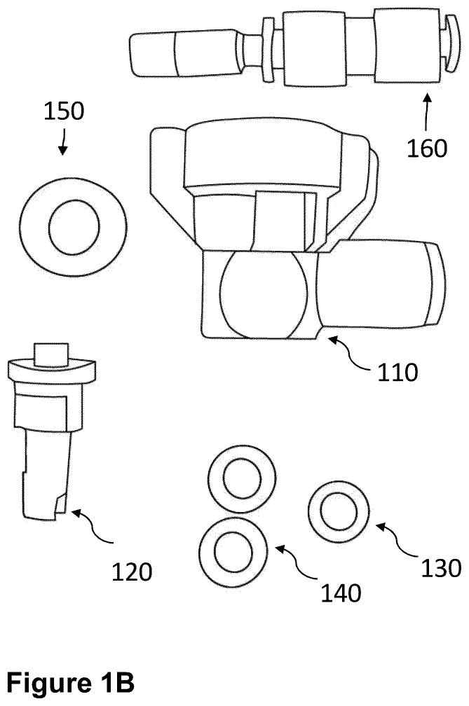

FIG. 2 illustrates two types of O-ring (130, 140). FIG. 2A depicts Type 1 O-ring (7.6 mm×2.4 mm, 130) (two of which are configured to fit inside at the bottom of the cap). FIG. 2B depicts a Type 2 O-ring (7 mm×2 mm, 140 (which is configured to fit onto the body of bubble jet of the spray nozzle (120)).

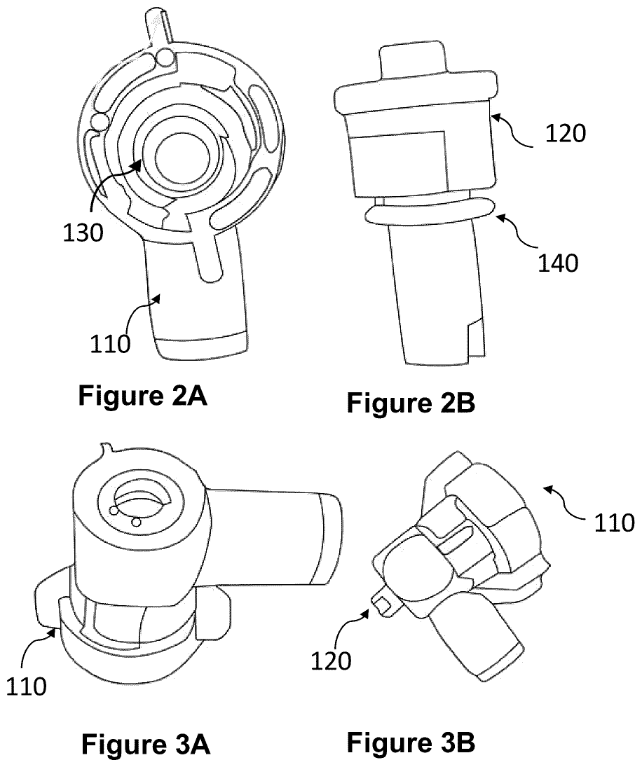

FIG. 3A illustrates the two O Rings inside the cap are configured to push past the hole that feeds the pipe. The O-ring on the body of the spray nozzle (FIG. 2B) is also configured to keep clear of the hole. FIG. 3B illustrates the spray nozzle (120) is configured to fit into the cap (110) aligned 90° to the lugs on the side of the cap.

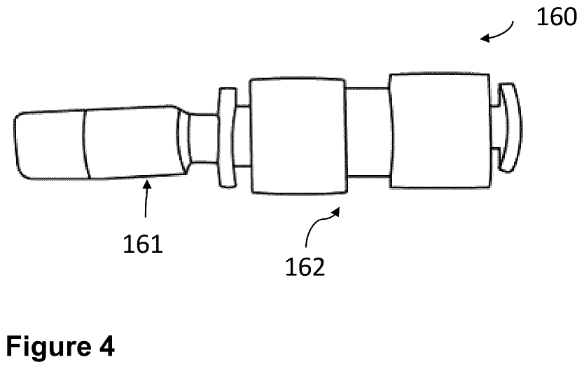

FIG. 4 illustrates an 8 mm to 6 mm adapter (162) and a 6 mm to 4 mm adapter (161) configured to feed a gas or liquid. However, other caps may be used using 6 mm banjo or any other suitable fittings.

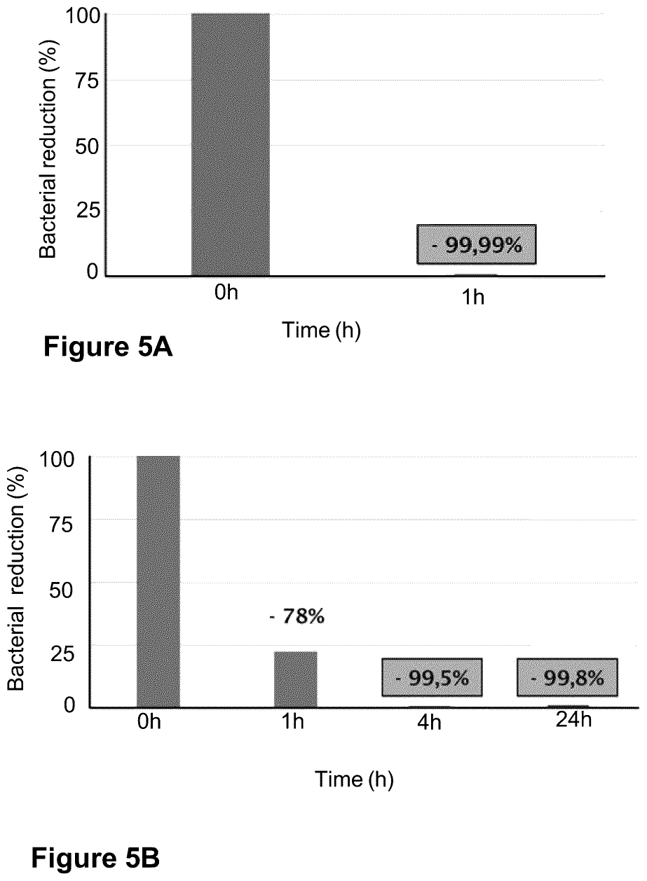

FIG. 5 illustrates tests on the anti-bacterial efficacy of a nano-coating developed for artificial pitches. Pieces of artificial pitch (10×10 cm) were coated with a water-based emulsion comprising 0.1% titanium dioxide nanoparticles and incubated with various bacteria-FIG. 5A, E. coli (ISO 22196), FIG. 5B, S. aureus (ISO 22196). The nano-coating leads to a strong anti-bacterial efficacy under humid conditions (LED-light, 400-800 nm; 37 C, 90% humidity).

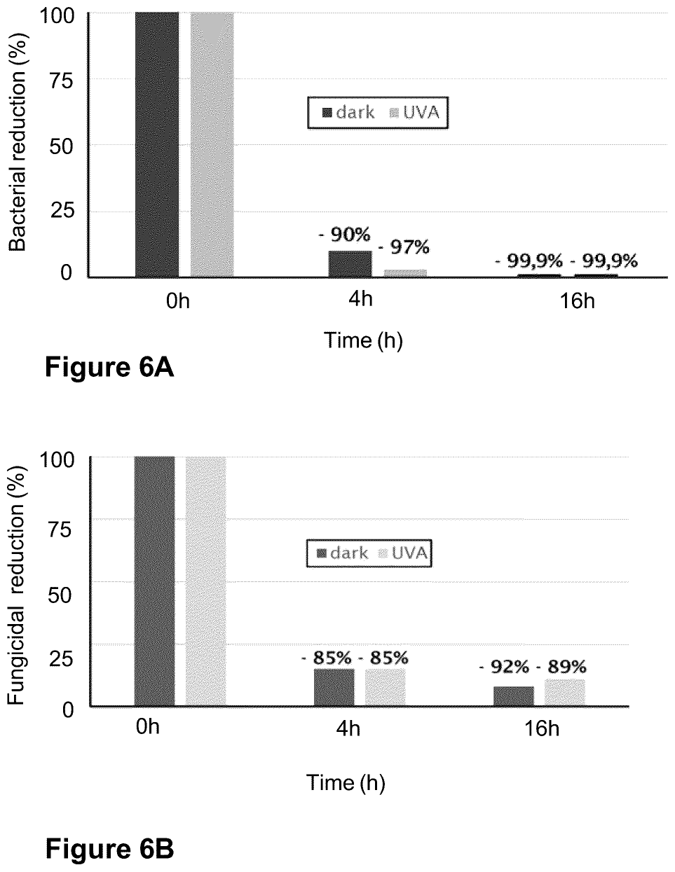

FIG. 6 illustrates tests on the anti-bacterial and fungicidal efficacy of the nano-coating developed for artificial pitches. Pieces of artificial pitch (10×10 cm) were coated with a water-based emulsion comprising 0.1% titanium dioxide nanoparticles and incubated with germ suspensions of Staphylococcus aureus and Aspergillus brasiliensis according to ISO 27447.

The nano-coating leads to a strong anti-bacterial and fungicidal efficacy under real outdoor conditions (either in the dark or under UVA-light (320-400 nm; 0.25 mW/sqcm; 25 C; 60% humidity).

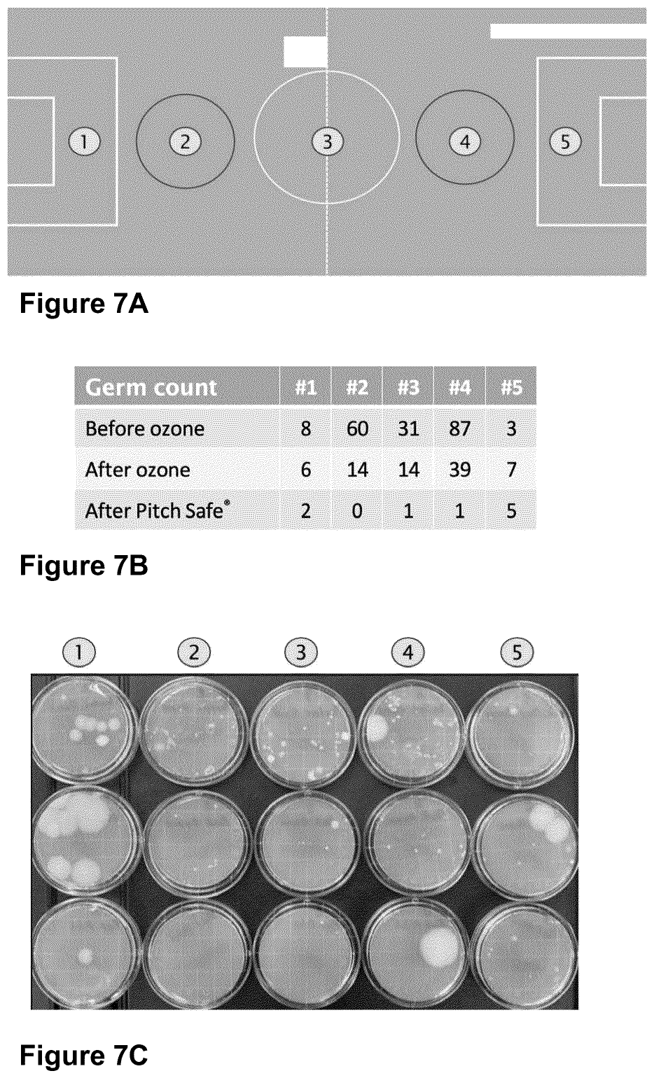

FIG. 7 illustrates field tests with preparation of a nano-coating after treating artificial grass with ozonated water. FIG. 7A shows definition of 5 sampling places (1-5) with agar plates. First sampling was taking before disinfection with ozone (10:30 to 10:40), second sampling after disinfection with ozone (11:00 to 11:20), third sampling after coating with the nano-coating developed for artificial pitches (11:40 to 11:50). The results (FIGS. 7B and 7C) reveal the central areas #2 to #4 show the highest pathogen and micro-organism load. Continuous pathogen and microorganism reduction was observed after treatment with ozone (6 ppm) and the nano-coating six months or more from the initial treatments.

### DETAILED DESCRIPTION

Further features of certain embodiments of the present invention are described below. Unless defined otherwise, all technical and scientific terms used herein have the meaning commonly understood by a person skilled in the art to which this invention belongs. Units, prefixes and symbols are denoted in their Système International de Unitese (SI) accepted form. Numeric ranges are inclusive of the numbers defining the range.

### Ozone

The invention provides compositions for controlling any pathogen as described herein, wherein the composition comprises ozone in combination with one or more pathogen reducing compound(s).

Ozone (or trioxygen) is an inorganic molecule with the chemical formula O₃. Ozone is a powerful oxidant, rendering it useful as a sterilizing and/or preserving agent in either aqueous or gas phase. For example, ozone is a powerful disinfectant commonly available for food sanitizing and water treatment.

The composition of the invention may comprise gaseous and/or liquid ozone. Typically, the composition comprises ozonated water. Preferably, the ozonated water comprises both gaseous ozone (O₃) and oxygen (O₂). Typically, the oxygen has a stabilised pH (e.g., using CO₂ gas).

In certain embodiments, the ozonated water comprises one or more acids. For example, citric acid or CO₂ may be used to lower pH and maintain ozone in the water for an increased duration of time.

The ozone of the composition may be obtained in any suitable way. A wide variety of different systems for producing ozone are commercially available.

Due to its tendency to break down quickly, ozone cannot be easily stored or transported. Typically, ozone is generated on sited by ozone generators (also called “ozonators”). Ozone is most commonly produced by the passage of dry, ambient air or pure oxygen either past a source of ultraviolet light or through an electrical discharge (e.g., corona discharge). The ozone is then injected or diffused into the treatment stream.

Typically, the ozone is prepared on-site using a system comprising an ozone generator within about 60 minutes, about 45 minutes, about 40 minutes, about 30 minutes or less of applying the ozone to the site of infection.

Where corona discharge is used to produce ozone, two electrodes may be separated by a dielectric and gas-filled gap. AC voltage may then be applied to the cell. The electrical discharge in the gas-filled gap creates free, energetic electrons that dissociate O₂ molecules into oxygen (O) atoms. These oxygen atoms are intermediates that then form ozone.

Portable ozone generators are commercially available. Typically, the generator is adapted to accommodate the ozone levels required for any particular application. For example, software can be used to program the ozone generator depending, for example, on the amount of ozone required.

As ozone can be decomposed by heating, temperature control of the process gas and heat removal are important factors in ozone generator efficiency. Typically, an array of water-cooled tubular cells is used. Typically, the generating capacity of an ozone generator is increased by enriching the air with oxygen.

Typically, the ozone generator produces a gaseous stream comprising a high concentration of ozone from oxygen, an oxygen-enriched gaseous stream, or air. Typically, the ozone generator is self-contained and/or portable. Preferably, a corona discharge ozone maker is used as this is currently the most efficient method of producing ozone.

Typically, the system for producing ozone comprises a holding vessel comprising water. For example, the system may comprise means for inputting the gaseous ozone to the holding vessel to produce ozonated water.

In certain embodiments, an oxygen-enriched gaseous stream is produced using an oxygen concentrator assembly. The ozone from oxygen, an oxygen-enriched gaseous stream or air may be introduced into a water stream or flow by any suitable means. For example, a venturi injector or any other suitable injection assembly may be used. A venturi injector may provide a source of suction which urges the ozone-containing gaseous stream from the ozone generator into the water stream or flow. The water may be passed through the venturi injector only once prior to dispensing the ozonated water onto the site of infection through an outlet assembly connected to the fluid passageway.

Prior to dispensing the ozonated water onto the site of infection, the ozonated water may be mixed or combined with one or more additional compounds such as those further described herein.

In certain embodiments, the ozone system includes a water tank, an oxygen generator, electric generator and ozone generator, a pump (e.g., venturi injector) for injecting gaseous ozone into recirculated water to form an ozone-water mixture. In addition, a pressure regulating subsystem may be provided for maintaining a consistent, regulated internal pressure of the aqueous stream as the stream is processed within the unit or system.

In certain embodiments, the ozone system includes an ozone analyser for sensing the amount of dissolved ozone in the holding vessel. Such an analyser may also be used to hold the dissolved ozone level at a constant level.

In certain embodiments, the ozone system includes a top access port. This may be configured to allow any undissolved ozone and oxygen to exit the water tank. Typically, the access port is connected to an ozone destruct unit which will remove ozone making the air exiting the system safe.

Any suitable amount of dissolved ozone may be used in the holding vessel. The amount of dissolved ozone to include in the system may depend on the flow rate used to deliver the ozonated water (e.g., litres per hectare) and/or the ultimate dosage of ozone (ppm) to be applied to the site of infection. Typically, for example, the generator is adapted to generate ozone in quantities of between about 2 to 200 g per hour.

The skilled person will understand that flow rates and/or dosage of ozone to apply to the site of infection may be optimized depending, for example, on the overall area to be treated (e.g., number of hectares), the type of pathogen to be treated (e.g., parasitic nematodes or the like) and site of infection (e.g., sports playing surface or type of agricultural crop or machinery).

In certain embodiments, the system may dispense ozonated water at a flow rate of about 350, 400, 450, 500, 550, 600 litres or more per hectare. Typically, a flow rate of about 350 litres per hectare is used to dispense ozonated water, for example, to treat grassed playing surfaces (e.g., professional football pitches, USGA golf pitches or the like). However, lower flow rates may be used to treat smaller pitches.

In certain embodiments, the ozonated water stream has an ozone concentration of at least about 0.001 ppm, about 0.1 ppm, about 0.2 ppm, about 0.3 ppm, about 0.4 ppm, about 0.5 ppm, about 1 ppm, about 2 ppm, about 4 ppm, about 5 ppm, about 6 ppm, about 8 ppm, about 10 ppm, about 20 ppm, about 30 ppm, about 40 ppm, about 50 ppm or more. For example, the ozonated water may preferably comprise at least about 10 ppm ozone.

In preferred embodiments, a valve delivers the ozonated water at a rate between about 0.001 ppm to about 50 ppm. The skilled person would understand the dosage of ozone may also depend on the type (and/or numbers) of pathogen to be treated, the size and/or type of pitch to be treated, or the like.

In certain embodiments, the system for dispensing ozone may comprise a spray assist assembly to a site of infection.

For example, a retractable spray lance or the like may be used to deliver the ozone and/or one or more additional compounds. Typically, the components of the system are sized and adapted to be mountable to a vehicle for transporting the system. For example, the vehicle may comprise spray arms and/or heads for delivering the composition of the invention.

In preferred embodiments, the system further comprises means of combining the ozone with one or more additional compounds (e.g., pathogen reducing compounds such as flavonoids or nano-coatings) as further described herein.

In certain embodiments, the ozonated water (or non-ozonated water within the same holding vessel) are mixed with one or more pathogen reducing compound(s) using a spray nozzle configured to combine flow from at least two separate streams. For example, a first stream may comprise the ozonated water (or non-ozonated water) and a second stream may comprise the pathogen reducing compounds.

In one embodiment, a spray nozzle is modified to include an air cap to mix the ozonated (or non-ozonated) water of a first stream passing through the spray nozzle with the pathogen reducing compounds of a second stream passing through the cap from a pressurised tank (see FIGS. 1 to 4). In this Example, the spray nozzle comprises a shrouded cap around it and the flow of a first stream through the nozzle may be used to draw in either air, gas or liquid via a second stream through the cap. This air, gas or liquid can be an additive to the ozonated (or non-ozonated) water already in the flow of the nozzle. Advantageously, this configuration prevents contamination of the ozonated water holding vessel of the first stream with the pathogen reducing compound(s) in the second stream.

### Pathogen Reducing Compounds

In certain embodiments, the composition further comprises one or more pathogen reducing compounds.

Any suitable pathogen reducing compounds may be used in the compositions and methods described herein. For example, the compound may have insecticide, fungicide, nematicide, bactericide, hydrophilic, photocatalytic and/or anti-viral properties.

Typically, the pathogen reducing compound is a naturally occurring compound. Typically, the pathogen reducing compound is highly effective in the control of many pests and pathogens as described herein. Typically, the pathogen reducing compound is present within a composition that boosts a plants' own defence system and/or alleviates the symptoms of stress and damage caused by an attack. Typically, the pathogen reducing compound is within a composition that is not designed to kill the pests but to deter and discourage them from attacking the plant.

In certain embodiments, the pathogen reducing compound is a natural nematicide. For example, the nematicide may be a garlic-derived polysulfide, neem-extract, root exudate of marigold (Tagetes) or carnivorous fungi (e.g., nematophagous fungi) or the like.

In certain embodiments, the pathogen reducing compound is an antioxidant. Such compounds act to inhibit oxidation, a chemical reaction that can produce free radicals and chain reactions that may damage the cells within turf grasses or other agricultural crops. For example, the antioxidant may comprise one or more ascorbates, tocopherols, reduced glutathione and its derivatives, cysteines (half cystines) or the like.

### Flavonoids

In some preferred embodiments, the pathogen reducing compound comprises one or more flavonoids. Advantageously, the use of flavonoids can increase the duration in which the ozone is effective against pathogens at a site of infection whilst still maintaining effective numbers of beneficial microbes. Thus, inclusion of flavonoids helps soil (or other sites of infection) recover from application of the ozone.

In certain embodiments, the flavonoids are in a composition further comprising cold pressed seaweed. Typically, the composition may comprise about 25% plant flavonoids and about 75% cold pressed seaweed.

Typically, the flavonoids are particularly effective against pathogens. By way of non-limiting example, the flavonoids may be particularly effective against parasitic nematodes as described herein. The flavonoids are typically also particularly effective against additional pathogens as described herein.

Flavonoids are phenolic compounds having the general structure of two aromatic rings connected by a three-carbon bridge. Flavonoids are produced by plants and have many functions, for example as beneficial signalling molecules and as protective agents against pathogens.

As used herein, the term “flavonoid” includes any flavonoid compound, isomer or salt thereof. The one or more flavonoids may be natural flavonoids, synthetic flavonoids or any combination thereof.

The flavonoids of the composition may be obtained in any suitable way. The flavonoids can be isolated from any suitable plant or seeds. Typically, the flavonoids are obtained from citrus or citrus waste (e.g., orange or peel) using techniques already described in the art (see, especially, “processing of citrus peel for the extraction of flavonoids for biotechnological applications”, in book: Flavonoids: Dietary sources, Properties and health Benefits (p443-459).

In certain embodiments, the flavonoids are extracted by solvent extraction (Xu et al., Journal of Agricultural and Food Chemistry (2007, 55 330-335); Zia-ur-Rehman, Food Chemistry (2006, 99:450-454); Anagnostopoulou et al., Food Chemistry (2006, 94 19-25); Li et al., Separation and Purification Technology (2006, 48:182-188); Jeong et al., Journal of Agricultural and Food Chemistry (2004, 52 3389-3393); Manthey and Grohmann, Journal of Agricultural and Food Chemistry (1996, 44 811-814), hot water extraction (Xu et al., 2007), alkaline extraction (Bocco et al., Journal of Agricultural and Food Chemistry (1998, 46 2123-2129; Curto et al., Bioresource Technology (1992, 42 83-87), resin-based extraction (Kim et al., Journal of Food Engineering (2007, 78 27-32); Calvarano et al., Perfumer and Flavorist (1996, 21 1-4), electron beam- and γ-irradiation-based extractions (Kim et al., Radiation Physics and Chemistry 2008, 77 87-91), supercritical fluid extraction (Giannuzzo et al., Phytochemical Analysis (2003, 14 221-223) or enzyme-assisted extraction (Puri et al., International Journal of Biological Macromolecules (2011, 48 58-62); Li et al., Separation and Purification Technology 2006, 48 189-196).

In alternative embodiments, the flavonoids are produced by genetically engineered organisms (e.g., yeast) as described, for example, in Rolston et al, Plant Physiology (Plant Physiology) 137:1375-88 (2005).

In preferred embodiments, the flavonoid is derived from citrus. For example, the flavonoids may comprise “Flav-X” or “SGS-Activate” as commercially available from SeeGrow Solutions Limited. In certain embodiments, the flavonoid is an anthocyanidin, flavan-3-ol, flavonol, flavanone, flavones, isoflavone or chalcone. In certain embodiments, the anthocyanidin is cyanidin, delphinidin, malvidin, pelargonidin, peonidin or petunidin. In certain embodiments, the flavan-3-ol is a proanthocyanidin, theaflavin, thearubigin, catechin, epicatechin, epigallocatechin, gallocatechin or a derivative thereof. In certain embodiments, the flavonol is isorhamnetin, kaempferol, myricetin, fisetin or quercetin. In certain embodiments, the flavone is apigenin, luteolin, baicalein or chrysin. In certain embodiments, the flavanone is eridictyol, hesperetin or naringenin. In certain embodiments, the isoflavone is daidzein, genistein, glycitein, Biochanin A or formonetin. In certain embodiments, the chalcone is naringenin or eriodictyol. In certain embodiments, the flavonoid is a phytoalexin, coumestrol, glyceollin which have been shown to increase resistance to, or minimize the effect of, nematode presence. In certain embodiments, the flavonoid is a glyceollin, phaseollin, sakuranetin, isoflavonoid, peterocarpan, medicarpin, coumesterol, psoralidin, quercetagetin, flavan-3,4-diol, condensed tannin, daidzein, genistein, kaempferol, quercetin, myricetin, patuletin, E-chalcone or any combination thereof.

The composition may comprise any suitable amount of flavonoid. The amount of flavonoid to be applied to a site of infection may depend, for example, on the overall area to be treated (e.g., number of hectares), the type of pathogen to be treated (e.g., parasitic nematodes or other pathogens) and particular site of infection (e.g., sports playing surface or type of agricultural crop).

In certain embodiments, the flavonoids are mixed with ozone prior to being dispensed to a site of infection. Any suitable type of mixing control or vessel may be used to combine ozone with flavonoids and/or other compounds as described herein. For example, commercially available Dosatron models (Dosatron International Inc., Florida) may be used to combine flavonoids (or other pathogen reducing compounds) with ozonated water. Any other suitable type of system may also be used to regulate and/or control the concentration of flavonoids and/or other compounds as described herein.

In certain embodiments, the level of flavonoids (or other pathogen reducing compounds) is set at a pre-determined level, and a control system (e.g., Dosatron or the like) is used to add the relevant flavonoid(s) or other compound(s) when its concentration falls below the pre-determined level.

The amount of flavonoid or other pathogen reducing compounds used in the composition may depend on the flow rate used to deliver the ozonated water and/or flavonoids or other compounds (e.g., litres per hectare) and/or the dosage of flavonoids or other compounds (mg/l) to be applied to the site of infection.

In certain embodiments, the composition comprises at least about 0.1 ppm, about 0.2 ppm, about 0.3 ppm, about 0.4 ppm, about 0.5 ppm, about 1 ppm, about 2 ppm, about 4 ppm, about about 5 ppm, about 6 ppm, about 8 ppm, about 10 ppm, about 20 ppm, about 30 ppm, about 40 ppm about 50 ppm or more flavonoids. For example, the composition may preferably comprise at least about 10 ppm flavonoids.

In preferred embodiments, a valve delivers the flavonoids at a rate between about 0.001 ppm to about 50 ppm. The skilled person would understand the dosage of flavonoids may also depend on the type (and/or numbers) of pathogen to be treated, the dosage of ozonated water, the size and/or type of pitch to be treated, or the like.

In certain embodiments, the system may dispense the flavonoids or other pathogen reducing compounds at a flow rate of about 1, 1.2, 1.3, 1.4, 1.5, 1.6, 1.7, 1.8, 1.9, 2.0 litres or more per hectare. Typically, a flow rate of about 2 litres per hectare is used, for example, to treat parasitic nematode infection of grassed playing surfaces as described herein.

Any suitable ratio of flavonoid: ozonated water may be used. Typically, a ratio of about 1:100, about 1:200, about 1:300, about 1:400, about 1:500, about 1:1000 flavonoids to ozonated water is used. By way of example, about 1 litre of flavonoids (e.g., FlavX which contains 3% flavonoids) may be used per 350 litres of ozonated water to treat grassed playing surfaces (e.g., professional football pitches, USGA golf pitches or the like). However, lower flow rates may be used to treat smaller pitches.

### Nano-Coatings

In some preferred embodiments, the pathogen reducing compound provides a nano-coating to the (e.g., artificial) pitch surface. Typically, the nano-coating is in the form of nanoparticles.

For example, the nano-coating may be a spherical nanoparticle, nanotube, mesostructured or the like. Typically, the nanoparticles are less than about 100 nm, e.g., about 50 nm, about 30 nm, about 10 nm or less. In preferred embodiments, the nanoparticles have an average particle size of about 10 nm.

Typically, the nano-coating has hydrophilic properties. Advantageously, this may facilitate binding of bacteria or other microorganisms. For example, the nano-coating may be acid-functionalised, have a positive charge and/or comprise silver ions.

Typically, the nano-coating is photocatalytic. For example, the nano-coating may disinfect the pathogen(s) under light illumination.

In some embodiments, the pathogen reducing compound comprises at least one metal oxide and/or metal salt. For example, the metal oxide or salt may be titanium (Ti), Zirconium (Zr) hafnium (Hf) and/or rutherfordium (Rf). Typically, the metal oxide is zirconium dioxide (i.e., zirconia), hafnium dioxide (i.e., hafnia) or titanium dioxide (i.e., titania).

In some embodiments, the metal oxide (e.g., titanium dioxide) is combined with an inorganic metal, non-metal and/or two-dimensional material. The inorganic metal may comprise, for example, copper, silver, manganese, or the like. The non-metal may comprise phosphorous, fluorine, calcium, or the like. The two-dimensional material may comprise Mxenes, MOF, graphdiyne or the like.

In some embodiments, the pathogen reducing compound is an aqueous based suspension comprising about 0.01% to about 10% of the metal oxide. For example, the pathogen reducing compound may comprise about 0.1% to about 2% of the metal oxide. In preferred embodiments, the pathogen reducing compound comprises about 0.1% of the metal oxide (e.g., titanium dioxide).

In some embodiments, the pathogen reducing compound comprises phosphorous and fluorine dope titanium dioxide (P/F-TiO₂). Preferably, the molar ratio of F/Ti and P/Ti is fixed at about 0.03.

The pathogen reducing compound may be synthesised by a sol-gel method, liquid-phase synthesis method or the like. Methods of synthesising nano-coatings are described, for example, by Kumaravel et al., Chemical Engineering Journal 416 (2021) 129071 herein incorporated by reference. Pathogen reducing compounds capable of providing anti-microbial or nano-coatings are well-described in the art and commercially available. For example, Kastus®, GermstopSQ, Green Millennium, NanoSeptic, Berger Elegance, GERM ARMOR®, TITANO®, TitanoClean®, DrivePur, PureTi, PALCCOAT, TOTO, Pilkington SaniTise™, and airlite are the approved photocatalytic products or coatings in the market for antimicrobial applications.

In some embodiments, the nano-coating is derived from TITANO®, as developed by HECESOL GmbH and Open World Technology Ltd.

In some embodiments, the pathogen reducing compound is a water-based suspension comprising titanium dioxide nanoparticles. The pathogen reducing compound may further comprise additional inorganic materials (e.g., silver chloride, silicum dioxide or the like). Synthesis routes to nanostructured titanium dioxides are well described in the art. See also, for example, Kartini, I. et al., 2018, ‘Nanostructured Titanium Dioxide for Functional Coatings’, in D. Yang (ed.), Titanium Dioxide-Material for a Sustainable Environment, IntechOpen, London. 10.5772/intechopen.74555 and US20070199480A1, both herein incorporated by reference.

In certain embodiments, the coating is stable on the pitch surface for up to about 1, 2, 3, 4, 5, 6, 7, 8, 9, 10, 11, 12 months or more.

In preferred embodiments, the coating has an isoelectric point of 3.4±0.2, average particle size of about 10 nm, specific surface area of about 130 m/g, and/or grain size of about 42±16 nm (e.g., as measured by atomic force microscopy (AFM)). Advantageously, the nano-coating may also have a higher number of contact points for microbial adhesion as compared to the commercial photocatalyst Degussa (Evonik) P25 TiO₂ (isoelectric point of 5.6±0.5, average particle size of 22 nm, specific surface area of 55 m2/g and/or grain size of 180±35 nm).

In certain embodiments, the system may dispense the nano-coating at a flow rate of about 1, 1.2, 1.3, 1.4, 1.5, 1.6, 1.7, 1.8, 1.9, 2.0, 10, 20, 30, 35 litres or more per hectare. Typically, a flow rate of about 2 litres per hectare is used, for example, to treat parasitic nematode infection of artificial playing surfaces as described herein.

Any suitable ratio of nano-coating: ozonated water may be used. Typically, a ratio of about 1:100, about 1:200, about 1:300, about 1:400, about 1:500, about 1:1000 nano-coating to ozonated water is used.

### Oxidizing Reagents

In certain embodiments, the composition of the invention may further comprise one or more additional oxidizing reagents. Such compounds can oxidize other substances.

Advantageously, the use of such oxidizing reagents can enhance the oxidizing activity of the ozone.

Any suitable oxidizing reagent may be used in the composition, method and systems as described herein.

In certain embodiments, the oxidizing reagent is a halogen. Typically, the oxidizing reagent is fluorine, chlorine, bromine, iodine, hypochlorite, chlorate, nitric acid, sulfur dioxide, hexavalent chromium, permanganate, manganate, ruthenium tetroxide, osmium peroxide, thallic compound or the like.

In preferred embodiments, the oxidizing reagent is oxygen. For example, the ozone (O₃) may be mixed with any suitable amount of pure oxygen (O₂) prior to dispensing the ozone to a site of infection.

In preferred embodiments, the oxidizing reagent is hydrogen peroxide. For example, the ozone (O³) may be mixed with any suitable amount of hydrogen peroxide prior to dispensing the ozone to a site of infection.

In certain embodiments, the oxidizing reagent includes both oxygen and hydrogen peroxide.

In certain embodiments, the levels of oxygen (O₂) and/or hydrogen peroxide are set at pre-determined levels, and a control system (e.g., Dosatron or the like) as already described above is used to add the relevant compound(s) when its concentration falls below the pre-determined level.

In certain embodiments, the one or more oxidizing reagents are mixed using a spray nozzle configured to combine flow from at least two separate streams as described herein.

### Additives

In certain embodiments, the composition of the invention further comprises one or more additives.

Any suitable additives may be used in the composition, method and systems as described herein.

In certain embodiments, the composition may further comprise a seaweed extract. In certain embodiments, the composition further comprises a neem and/or garlic extract.

Advantageously, the use of such additional agents may further enhance the efficacy of the composition in controlling pathogens (e.g., nematodes). In certain embodiments, the composition may further comprise any one or more of ascorbic acid, Vitamin P, acitic acid, glycerine and glycine betaine.

In certain embodiments, the composition of the invention comprises one or more surfactant. For example, the composition may include an anion, cationic and/or non-ionic surfactant including, but not limited to, aliphatic sulfonic ester salts like lauryl sulfate, aromatic sulfonic acid salts, salts of lignosulfates, and soaps. Examples of nonionic surfactants are the condensation products of ethylene oxide with fatty alcohols such as oleylalcohol, alkyl phenols, lecithins, and phosphorylated surfactants, such as phosphorylated ethylene oxide/propylene oxide block copolymer and ethoxylated and phosphorylated styryl-substituted phenol. Additional surfactants are anionic wetting agents, such as sodium salts of sulfated alkyl carboxylate, and/or alkyl naphtalenesulphonate, and/or dispersing agents such as naphthalene formaldehyde condensate.

In certain embodiments, the composition of the invention comprises one or more vitamins and/or minerals. Examples of vitamins for use in the composition include but are not restricted to the following: biotin, folic acid, vitamins B, B2, B3, B6, B7, B12, C, and K. Examples of minerals include any mineral that can enhance the growth of the plant and/or beneficial bacteria. Specific examples of minerals include potassium, iron, sulfur, magnesium, boron, manganese, zinc or the like.

In certain embodiments, the composition of the invention includes at least one sticking agent. A sticking agent is a compound that increases the length of time that at least one other component in the composition (e.g., ozone and/or flavonoids or other compounds) stays in contact with another component of the composition and/or with the soil, plant part or other material the composition is being applied. Suitable sticking agents include, but are not limited to, yucca plant extract, and clays such as Kaolin clay, fine benign hygroscopic powders and the like.

In certain embodiments, the composition of the invention includes at least one additional compound that further extends the time period over which the composition remains effective. Compounds for extending the effective period of a composition include at least one compound selected from the group consisting of aluminum silicate, fine clays, Kaolin clay, aluminum oxide, zinc oxide, and the like.

In certain embodiments, the one or more additives are mixed using a spray nozzle configured to combine flow from at least two separate streams as described herein.

### Pathogen Control

The invention further provides a method of controlling one or more pathogens comprising delivering an effective amount of ozone onto the surface of a grassed, artificial or hybrid pitch.

The invention also provides methods of controlling one or more pathogens comprising delivering an effective amount of ozone and pathogen reducing compounds (e.g., flavonoids or nano-coatings) to other sites of infection including for example, agricultural crops or machinery as described herein.

Any suitable pathogen (e.g., pest) or disease may be controlled (or treated) using compositions and methods as described herein. Typically, the pathogen is a plant pathogen.

As used herein, the term “pathogen” includes any organism that causes infectious disease of a plant and/or has a negative effect on the growth of a plant directly or indirectly. For example, the pathogen may be a fungi, nematode, oomycete, bacteria, virus, viroid, virus-like organisms, phytoplasma, protozoa or parasitic plants.

In certain embodiments, the pathogen is a bacteria, e.g., cocci or rod-shaped bacterium.

In certain embodiments, the pathogen is Streptococcus or Staphylococcus aureus. For example, the pathogen may be methicillin-resistant Staphylococcus aureus (MRSA). Such bacterial infections may be particularly problematic with respect to artificial playing surfaces as described herein. Certain embodiments of the invention relate to compositions and methods for treating bacterial infection (e.g., MRSA) of artificial playing surfaces. Advantageously, the compositions and methods described herein can make artificial playing surfaces safer to use, e.g., reduce the risk of any player developing an infection in case of any cut or abrasion to the skin during activity on the artificial playing surface.

In certain embodiments, the pathogen is a soil pathogen. The pathogen may cause turfgrass diseases such as leaf spot, red thread, rust, anthracnose, patch (e.g., take-all, brown), ring spot (e.g., necrotic ring spot), dollar spot, root rot, mould or the like. Such pathogens may be particularly problematic with respect to grassed or hybrid playing surfaces as described herein. For example, the pathogen may be a fungus such as fusarium, leaf spot fungus or the like.

The pathogen may be an insect such as leatherjacket (crane fly), fever fly, chafer, Japanese beetle (Popilla Japonica) or other turf grass insect such as white grub, sod web worms, army worms, mole crickets, bill bugs or the like.

In certain embodiments, the pathogen is a nematode, e.g., a parasitic nematode. The nematode may be of the order Tylenchida. The nematode may be a root-knot nematode (e.g. Meloidogyne sp.), lesion nematode (e.g. Pratylenchus sp.), cyst nematode (e.g. Heterodera sp.), dagger nematode (e.g. Xiphinema sp.), stem and bulb nematode (e.g. Ditylenchus sp.) or the like.

In certain embodiments, the nematode is a root-knot nematode. For example, the nematode may be a parasitic nematode such as Meloidogyne incognita, H. glycines, B. longicaudatus, H. contortus, A. suum, B. malayi or the like.

As used herein, the term “controlling” refers to preventing a pathogen or disease from infecting or affecting a plant (e.g., turf grass or other crop) or other site of infection (e.g., artificial sports pitch, agricultural and/or ground care machinery or the like). The ozone and/or flavonoids (or other pathogen reducing compounds) may act, for example, as a biocide, bactericide, bacteriostat, fungicide, fungistat, insecticide and/or may interfere with one or more functions of a given pathogen that enables it to infect a given plant under a given set of environmental conditions.

In certain embodiments, the invention provides a method of reducing the viability or fecundity or slowing the growth or development or inhibiting the infectivity of one or more pathogens.

In certain embodiments, the invention provides a method of controlling a pathogen population, wherein the method comprises delivering an effective amount of ozone and one or more pathogen reducing compounds (e.g., flavonoids or nano-coatings) to a site of infection.

In certain embodiments, the invention provides a method of protecting the surface of a grassed, artificial or hybrid pitch surface, wherein the method comprises delivering an effective amount of ozone and one or more flavonoids or other pathogen reducing compounds to a site of infection.

In certain embodiments, the invention provides a method of improving the yield of one or more crop plants, wherein the method comprises delivering an effective amount of ozone and one or more flavonoids or other pathogen reducing compounds to a site of infection.

In certain embodiments, the method of controlling one or more pathogens comprises reducing the amount of damage done by a pathogen to a plant (e.g., turf grass or other crop) relative to a control plant that is likewise infected with the pathogen but not exposed to the composition.

In certain embodiments, the methods of the invention further comprise determining the number and/or type of pathogens in a sample after treatment with the ozone and/or flavonoids or other pathogen reducing compounds.

In certain embodiments, the number and/or type of pathogens in the sample are compared to a control sample obtained prior to the treatment. Alternatively, the number and/or type of pathogens in the sample may be compared to reference levels or a reference index as further described herein.

The determination of the number and/or type of pathogens in the sample may allow a subsequent treatment strategy to be determined. For example, if the number of pathogens after treatment are above a threshold level, a second treatment with ozone and/or pathogen reducing compounds (e.g., flavonoids) (optionally at an increased concentration) may be applied to the site of infection. Alternatively, a different type of treatment (e.g., alternative pesticide or other treatment) may be applied to the site of infection.

Conversely, if the number of pathogens after treatment are below a threshold level, no further treatment may be required.

Any suitable technique may be used to determine the number and/or type of pathogens in the sample. The technique chosen may depend on the type of pathogen(s) being quantified.

In one non-limiting embodiment, the pathogens are parasitic nematodes. In such embodiments, the rootzones may be extracted from soil samples to determine the presence of any plant parasitic nematodes and the washed roots may be assessed visually for parasitic nematode species. Typically, the total number of nematodes in the rootzone sample may be determined and the populations of the parasitic species recorded.

In such embodiments, a nematode damage index (NDI) may provide an indication of the overall level of nematode-induced stress within the turf. The NDI may take account of the individual threshold value for each species (i.e., the population that is likely to cause significant damage to the turf) and the recorded population of each species.

For example, an NDI of about 0.0 to 0.5 may typically indicate that treatment may not be required but levels of plant parasitic nematodes should be monitored. An NDI of about 0.5 to 1.0 may typically indicate that treatment should be considered to restrict nematode levels building to damaging levels. An NDI of between about 1.0 to 10 may typically indicate nematode levels may be approaching damaging levels. An NDI of about 10 or more may typically indicate that treatment is required.

In such embodiments, a “threshold level” as described herein may be a nematode damage index of about 10.

In one non-limiting embodiment, the pathogen are bacteria and/or fungi. In such embodiments, a sample comprising the bacteria and/or fungi may be analysed. For example, the total number and/or type of bacteria and/or fungi may be determined in the sample using any suitable technique.

In such embodiments, the amount of bacteria and/or fungi may provide an indication of the overall level of pathogen-induced stress within the turf. For example, at least about 150 μg, 175 μg, 200 μg per ml or more may indicate a high level of bacteria in the sample. Conversely, at least about 150 μg, 100 μg, 75 μg per ml or less may indicate a low level of bacteria in the sample.

Typically, an “effective amount” of ozone and/or pathogen reducing compound as described herein is an amount sufficient to reduce bacteria in a sample to about 100 μg or less. An effective amount of the ozone and/or pathogen reducing compound may also take account of the type of bacteria in the sample.

In certain embodiments, the ozone and/or pathogen reducing compounds are applied in an amount effective to achieve a desired ratio of fungi to bacteria. For example, both beneficial bacterial and fungi need to be present in order for nutrient cycling to occur in soil. The ratio of fungi to bacteria may be optimised in soil following the methods of the invention.

In one embodiment, the ozone and/or pathogen reducing compounds are applied in an amount effective to achieve a ratio of fungi to bacteria of about 0.1 to 1. Typically, such ratios are preferred for crop plants such as weedy stage or irrigated wheat.

In one embodiment, the ozone and/or pathogen reducing compounds are applied in an amount effective to achieve a ratio of fungi to bacteria of about 0.3 to 1. Typically, such ratios are preferred for early successional plants (e.g., early annuals such as dryland wheat or bromus, Bermuda, brassicas, mustard and kale crops).

In one embodiment, the ozone and/or pathogen reducing compounds are applied in an amount effective to achieve a ratio of fungi to bacteria of about 0.75 to 1 to about 0.8 to 1. Typically, such ratios are preferred for mid succession grasses, vegetables, herbs and forbes.

In one embodiment, the ozone and/or pathogen reducing compounds are applied in an amount effective to achieve a ratio of fungi to bacteria of about 1 to 1. Typically, such ratios are preferred for late successional grasses, productive row crops, pastures, turf, prairies (fescues, corn, wheat, lucerne).

In one embodiment, the ozone and/or pathogen reducing compounds are applied in an amount effective to achieve a ratio of fungi to bacteria of about 2 to 1 to about 5 to 1. Typically, such ratios are preferred for fruit bushes.

In one embodiment, the ozone and/or pathogen reducing compounds are applied in an amount effective to achieve a ratio of fungi to bacteria of about 5 to 1 to about 100 to 1. Typically, such ratios are preferred for deciduous trees and/or orchards.

### Spray Delivery

The ozone and/or pathogen reducing compounds (e.g., flavonoid(s)) may be delivered by spraying to any suitable site of infection.

In certain embodiments, ozone and one or more pathogen reducing compounds (e.g., flavonoids) are delivered to an agricultural crop.

The composition of the invention may be applied to any suitable crop. For example, the compositions may be applied to any monocot or dicot plant, depending on the pathogen control desired. Exemplary plants include, but are not limited to, alfalfa, banana, beans (e.g., soybean), peas, cereals (e.g., barley, wheat, rye), chickpea, citrus, clover, corn, cotton, grapes, grasses, peanut, potato, rice, small fruits, soybean, sugar beet, sugar cane, tobacco, tomato, cucumber, pepper, carrots, rapeseed (canola), sunflower, safflower, sorghum, strawberry, banana, turf, ornamental plants or the like.

In alternative embodiments, the ozone and one or more pathogen reducing compounds (e.g., flavonoids) are delivered to agricultural machinery (e.g., tractors or the like) and/or grounds care equipment (e.g., lawnmowers or the like) known or suspected of being infected with one or more pathogen(s).

In certain embodiments, ozone is delivered onto the surface of a grassed, artificial or hybrid pitch. For example, ozone and one more pathogen reducing compound(s) may be applied to any turf for playing sport, for recreation and/or for ornamental purposes. Typically, the turf can be used as a field for playing sport such as football (soccer), tennis, hockey, American football, golf, athletics, rugby, baseball or any other sport that can be played on turf grass. Typically, the golf turf is USGA standard.

As described herein, artificial turf typically comprises a dense cover of polymeric fibres of a defined length on which a filler material consisting of sand or rubber or the like of specified granulometry is distributed.

As described herein, a hybrid pitch surface typically comprises a combined system of mixed natural and artificial turf.

As described herein, the ozone is typically prepared on-site by electrolysis prior to delivery. For example, the ozone may be obtained using an ozone system as discussed above. Preferably, the ozone is applied to the site of interest within about 1 hour, 40 minutes, 30 minutes or less after being produced.

In certain embodiments, the pathogen reducing compound (e.g., flavonoids or nano-coatings) are mixed directly with a solution of ozonated water. Any suitable technique may be used to mix pathogen reducing compounds such as flavonoids with ozonated water. As described herein, commercially available systems include “Dosatron” or the like which enable any further liquid components to be injected in a controlled manner to a holding vessel comprising the ozonated water. In such embodiments, ozone and the pathogen reducing compounds may be administered to the site of infection at the same time.

Alternatively, flavonoids (or any other pathogen reducing compounds as described herein) may not be premixed with the ozonated water. Instead, the flavonoids or other compounds may be administered separately at any suitable time point before or after the delivery of ozone to the site of infection. Typically, the flavonoids or other compounds are administered shortly before or after ozone treatment, e.g., there may be a delay of about 1, 2, 3, 4, 5, 7, 14, 21, 28 days or less between treatment with the ozonated water and the flavonoids or other compounds (e.g., nano-coatings).

In some embodiments, the pathogen reducing compound(s) (e.g., flavonoids or nano-coatings) are mixed with the ozonated (or non-ozonated) water using a modified spray nozzle as described herein.

Any suitable technique may be used to deliver ozonated water and/or flavonoids or other pathogen reducing compounds to a site of infection. For example, compositions of the invention may be delivered via spraying, saturation via an irrigation system or the like. Such delivery systems are well described in the art and well-known to the skilled person. Typically, the delivery systems are modified to comprise ozone-compatible materials.

In certain embodiments, the composition is applied to the aerial parts of plants (e.g., shoots, leaves, flowers or the like).

Typically, the composition of the invention is liquid (e.g., comprises ozonated water). The spraying of liquid compositions may be accomplished by a variety of methods including, but not limited to, blast sprayers, hose reel and handgun, walking sprays, aerial sprays or the like.

Typically, the components of the spray are modified to comprise ozone-resistant materials.

As described herein, an ozone-resistant material includes any material that does not degrade (or only degrades slowly) in the presence of ozone. Typically, an ozone-resistant material is a material where ozone has no effect and will last indefinitely in the presence of ozone. However, ozone resistant materials may also include materials where ozone only has a minor effect. Prolonged use with high concentrations of ozone may break down or corrode such materials, but they may still be utilised in the present invention.

In preferred embodiments, the ozone-resistant material comprises any one or more of Santoprene, Silicone, Stainless steel (304/316), Titanium, Polycarbonate, Butyl, Chemraz, CPVC, Cross-Linked Polyethylene (PEX), Durachlor-51, EPR, Ethylene-Propylene, Fluorosilicone, Glass, Hastelloy-C®, HDPE, Inconel, Kalrez, Kel-FR (PCTFE), PEEK, Polycarbonate, Polyurethane, PTFE, PVC, PVDF (Kynar®), Santoprene, Silicone, Vamac, Viton or the like. Ozone has no effect on these materials, they will last indefinitely.

In certain embodiments the ozone-resistant material comprises any one or more of EPDM, ABS plastic, Acrylic (Perspex®), Brass, Bronze, Copper, Flexelene, LDPE, Polyacrylate, Polyethelyne, Polysulfide, Stainless Steel (other grades), Tygon, Aluminium or the like. Ozone only has minor effect on these materials.

Any suitable spray rigs may be used to deliver ozone, flavonoids and/or any additional compounds as described herein. For example, the spray rig may be similar in construction to spray rigs conventionally used for treating crops with liquid chemicals. The spray rig may be chosen so it doesn't have any significant detrimental effect on the ozone being dispensed on the crop being treated. For example, the spray rig may provide a high-volume, low-pressure air flow which is mixed with the ozonated water stream (and optionally also flavonoids or other compounds as described herein) prior to dispensing the ozonated water to the crop. The spray rig is typically chosen or adapted to comprise ozone resistant materials as described herein.

In certain embodiments, the spraying system is tractor mounted, knapsack, walkover type, droplet applicator (CDA) or the like.

In certain embodiments, the surface of the site of infection (e.g., grass turf or the like) is aerated and/or punctured with holes prior to being sprayed. Advantageously, this allows the ozonated water and/or pathogen reducing compounds (e.g., flavonoids or nano-coating) to become saturated into the soil.

### Modified Spray Cap

Certain embodiments of the invention provide methods using spray cap apparatus configured to deliver an effective amount of the ozonated or non-ozonated water and pathogen reducing compound(s) by spraying onto a grassed, artificial or hybrid pitch surface. For example, the apparatus may comprise:
(a) A spray nozzle (120) configured to deliver a first stream of gas or liquid (e.g., ozone or non-ozone) by spraying;
(b) an air measurement cap (110) configured to draw a second stream of gas or liquid (e.g., comprising the pathogen reducing compound(s)) into the first stream.

Typically, the apparatus is formed of ozone-resistant material as described herein.

### Embodiments of the Invention

The present invention further comprises the subject matter of the following numbered paragraphs:

1. A composition for controlling pathogens, wherein said composition comprises ozone and one or more pathogen reducing compounds.

2. The composition of paragraph 1, wherein the pathogen reducing compound(s) comprise one or more flavonoid(s) and/or nano-coatings as described herein.

3. The composition of paragraph 1 to 2, wherein the composition comprises one or more additional oxidizing reagents.

4. The composition of any one of paragraph 1 to 3, wherein the oxidizing reagents are oxygen (O₂) and/or hydrogen peroxide.

5. A method of controlling one or more pathogens comprising delivering an effective amount of ozone and one or more pathogen reducing compounds to a site of infection.

6. The method of paragraph 5, wherein the pathogen reducing compound(s) comprise one or more flavonoid(s) and/or nano-coatings as described herein.

7. The method of paragraph 6, further comprising delivering one or more additional oxidizing reagents.

8. The method of paragraph 7, wherein the oxidizing reagents are oxygen (O₂) and/or hydrogen peroxide.

9. The method of any one of paragraph 5 to 8, wherein the pathogens are fungi, insect, nematode, oomycete, bacteria, virus, viroid, virus-like organisms, phytoplasma, protozoa and/or parasitic plants.

10. The method of paragraph 9, wherein the pathogen is a parasitic nematode.

11. The method of any one of paragraph 5 to 10, wherein the ozone and pathogen reducing compounds are delivered to soil, a grassed, artificial or hybrid pitch surface, an agricultural crop and/or machinery.

12. The method of any one of paragraph 5 to 11, wherein the ozone is prepared on-site by electrolysis prior to delivery.

13. The method of any one of paragraph 5 to 12, wherein the ozone and pathogen reducing compounds are delivered by spraying.

14. A method of controlling one or more pathogens comprising delivering an effective amount of ozone onto the surface of a grassed, artificial or hybrid sports pitch.

15. The method of paragraph 14, further comprising delivering an effective amount of one or more pathogen reducing compounds onto the surface of the grassed, artificial or hybrid sports pitch.

16. The method of paragraph 15, wherein the pathogen reducing compound(s) comprise one or more flavonoid(s) or nano-coating as described herein.

17. The method of any one of paragraph 14 to 16, further comprising delivering an effective amount of one or more additional oxidizing reagents onto the surface of the grassed, artificial or hybrid sports pitch.

18. The method of paragraph 17, wherein the oxidizing reagents are oxygen (O₂) and/or hydrogen peroxide.

19. The method of any one of paragraph 14 to 18, wherein the pathogens are fungi, insect, nematode, oomycete, bacteria, virus, viroid, virus-like organisms, phytoplasma, protozoa and/or parasitic plants.

20. The method of paragraph 19, wherein the pathogen is a parasitic nematode.

21. The method of any one of paragraph 14 to 20, wherein the ozone is prepared on-site by electrolysis prior to delivery.

22. The method of any one of paragraph 14 to 21, wherein the ozone and/or pathogen reducing compounds are delivered via spraying.

23. The method of any one of paragraph 14 to 22, wherein at least about 0.001 to about 50 ppm of ozonated water and/or pathogen reducing compound is delivered onto the surface of the pitch.

24. The method of any one of paragraph 5 to 23, wherein the method further comprises determining the number of pathogens in a sample obtained from the site of infection after treatment with the ozone and/or pathogen reducing compounds.

25. The method of paragraph 24, wherein, if the number of pathogens after treatment are above a threshold level, a second amount of ozone and/or pathogen reducing compounds are applied to the site of infection.

26. The method of paragraph 25, wherein the pathogen reducing compound comprises one or more flavonoids.

27. The method of paragraph 25 or 26, wherein the number of nematodes is determined, and the threshold level is a nematode damage index (NDI) of about 10.

28. The method of paragraph 27, wherein the number of bacteria and/or fungi is determined.

29. The method of any one of paragraph 14 to 28, wherein the ozone and/or pathogen reducing compounds are applied in an amount effective to achieve a ratio of fungi to bacteria of about 0.5 to about 1.5.

### EXAMPLES

In the following, the invention will be explained in more detail by means of non-limiting examples of specific embodiments.

### Example 1—Ozonated Water Targets Pathogenic Nematodes in Sports Turf

All soils contain nematodes. Most are beneficial bacterial or fungal feeding species which contribute positively to the soil environment and can be indicators of good soil health. However, parasitic nematodes may feed on plants and can attack turfgrass in two main ways:
Ectoparasites—these nematodes live in the soil, feeding externally on plant root cells. This can cause reduced root function and abnormal root morphology.
Endoparasites—these nematodes live for much of their life cycle inside plant roots, where they feed on root cells. Endoparasites usually cause major morphological and physiological abnormalities in the roots. For example, root knot nematodes (Meloidogyne) are particularly damaging endoparasites and their feeding causes severe root galling and loss of root function. Affected turf will be shallow rooted, chlorotic and will lose leaf density.

The nematode damage index (NDI) provides an indication of the overall level of nematode-induced stress within the turf. An NDI greater than 10 indicates that nematode damage symptoms are likely to be currently visible on the turf.

A premier league football (soccer) pitch infested with nematodes was treated with ozonated water. About 350 litres ozonated water was applied per hectare of the football pitch. The surface of the pitch was punctured by small holes and the ozonated water was applied directly into the soil.

The ozonated water was generated on site using a portable ozone generator supplied by Ozone Industries Ltd. Inside the ozone generator, ozone is produced from oxygen present in the feed gas by means of a silent electric plasma. Ozone is dissolved into the water in a mixing tank holding vessel.

This mix was constantly agitated, and the level of ozone required pre-set by the operator and maintained continuously. Typically, at least about 2 ppm, 4 ppm, 6 ppm, 8 ppm, 10 ppm or more ozone is used. The ozonated water was then applied across the surface of the pitch and directly into the soil.

Turf samples were obtained from the football pitch before and after treatment with the ozonated water. The rootzones were extracted to determine the presence of any plant parasitic nematodes and the washed roots were assessed visually for parasitic nematode species by a plant pathologist.

A simplified Baermann funnel method was used for extracting active nematodes from a known volume of the received rootzone sample. The total number of nematodes in the rootzone sample was determined and the populations of the parasitic species recorded. All non-parasitic nematodes present in the sample are recorded as “bacterial/fungal” species.

The results of the analysis are presented in Tables 1 and 2 below.

TABLE 1
summary of football pitch trials

| | Before | After | | percentage |
|---|---|---|---|---|
| Species | treatment | treatment | variation | variation |
| Meloidogyne | 3487 | 1335 | (2152) | −61.71% |
| J2s + males | | | | |
| (root-knot) | | | | |
| Beneficial | 2897 | 6617 | 3720 | 128.41% |
| microbes | | | | |

Advantageously, application of ozonated water to the soil led to a significant reduction in parasitic nematodes without substantially decreasing the numbers of beneficial microbes (Table 1).

Table 2 demonstrates the application of ozonated water to the sports pitch was capable of reducing the nematode damage index (NDI) from 21.3 to 8.0 (north samples) or 50.6 to 6.5 (south samples).

### Example 2—Flavonoids Enhance Longevity of the Ozone Treatment

A USGA standard golf course was treated with ozonated water.

About 350 litres of ozonated water was applied per hectare of the golf course. The ozonated water was premixed with about 2 litres of flavonoids per hectare prior to application of the mixture to the surface of the golf course. The surface of the pitch was punctured by small holes and the ozonated water was applied directly into the soil.

Ozone was generated on site using a portable ozone generator as described in Example 1. Inside the ozone generator, ozone is produced from oxygen present in the feed gas by means of a silent electric plasma. Ozone is dissolved into the water in a mixing tank holding vessel.

This mix was constantly agitated, and the level of Ozone required pre-set by the operator and maintained continuously. A Dosetron was included alongside the mixing tank, to enable flavonoids (“Flav-X”, commercially available from Seegrow Solution Limited) to be injected in a controlled manner to the premixed solution of ozonated water. The mixture of ozonated water and flavonoids was then sprayed across the surface of the pitch and into the soil via the punctured holes.

Analysis of the ozone treatment was performed over 272 days of a field trial on the golf course. The ozonated water/flavonoid mixture was applied to the turf as described above on days 178, 203 and 229 of the study. Samples were taken on Days 1, 202 and 272 of the field trial.

The rootzones of the samples were extracted to determine the presence of any plant parasitic nematodes and the washed roots were assessed visually for parasitic nematode species as described in Example 1 above.

Nematode numbers found in samples following the trial is presented in Table 3 below. Unexpectedly, the results show that the flavonoid/ozone mixture was capable of maintaining a Nematode Damage Index (NDI) less than 10 (a reduction of 32% for the start of the study) for 6 weeks and more after the final treatment. These results demonstrate that the combination of ozone with flavonoids is more effective and longer lasting as compared to treatment with ozone or flavonoids alone.

TABLE 2
Number of nematodes (per 100 ml rootzone) recorded in the football pitch sample

| | Endo | North | North | South | South | |
|---|---|---|---|---|---|---|
| | or Ecto- | before | after | before | After | Threshold |
| Nematode Species | parasite | treatment | treatment | treatment | treatment | Value |
| Bacterial/Fungal | | 14200 | 6538 | 13721 | 5805 | Not |
| Non-parasitic nematode species | | | | | | relevant |
| (beneficial) | | | | | | |
| Tylenchus | Ecto- | | | | | 300 |
| Heterodera J2s + males (cyst) | Endo- | | | | | 40 |
| Punctodera J2s + males (cyst) | Endo- | | | | | 100 |
| Hoplolaimus (lance) | Ecto- | | | | | 150 |
| Pratylenchus (lesion) | Endo- | | | | | 100 |
| Longidorus (needle) | Ecto- | | | | | 20 |
| Paratylenchus (pin) | Ecto- | | | | | 300 |
| Criconemella (ring) | Ecto- | | | | | 600 |
| Meloidogyne J2s + males (root- | Endo- | 230 + 33 | 114 | 995 | 131 | 20 |
| knot) | | males | | | | |
| Subanguina J2s (root gall) | Endo- | | | | | 80 |
| Hemicycliophora (sheath) | Ecto- | 625 | 180 | 69 | | 80 |
| Helicotylenchus (spiral) | Endo-/ | | | | | 400 |
| | Ecto- | | | | | |
| Rotylenchus (spiral) | Endo-/ | | | | | 500 |
| | Ecto- | | | | | |
| Paratrichodorus (stubby-root) | Ecto- | 33 | | | | 100 |
| Tylencorhynchus (stunt) | Ecto- | | | | | 300 |
| Pratylenchoides | Endo- | | | | | 80 |
| Xiphinema (dagger) | Ecto- | | | | | 100 |
| Belonolaimus (sting) | Ecto- | | | | | 10 |
| Gracilacus (pin) | Ecto- | | | | | 300 |
| Aphelenchoides | Endo-/ | | | | | 300 |
| | Ecto- | | | | | |
| Ditylenchus | Endo-/ | | | | | 400 |
| | Ecto- | | | | | |
| Heterodera cysts | Endo- | | | | | 40 |
| Meloidogyne galls | Endo- | | | | | 20 |
| Subanguina galls | Endo- | | | | | 20 |
| Nematode damage index (NDI) | | 21.3 | 8.0 | 50.6 | 6.5 | |

TABLE 3
Golf course trial summary

| | Endo- | Threshold | | | | |
|---|---|---|---|---|---|---|
| | or Ecto | for | | | | Percentual |
| Nematode Species | parasite | damage | Day 1 | Day 202 | Day 272 | Difference % |
| Tylenchus | Ecto- | 300 | | | | |
| Heterodera J2s + males (cyst) | Endo- | 40 | 237 | 0 | 0 | −100.00% |
| Punctodera J2s + males (cyst) | Endo- | 100 | | | | |
| Hoplolaimus (lance) | Ecto- | 150 | | | | |
| Pratylenchus (lesion) | Endo- | 100 | | | | |
| Longidorus (needle) | Ecto- | 20 | 6 | 6 | 5 | −16.67% |
| Paratylenchuus (pin) | Ecto- | 300 | | | | |
| Criconemella (ring) | Ecto- | 600 | | | | |
| Meloidogyne J2s + males (root-knot) | Endo- | 20 | | | | |
| Subanguina J2s (root-gall) | Endo- | 80 | | | | |
| Hemicycliophora (sheath) | Ecto- | 80 | | | | |
| Helicotylenchus (spiral) | Endo-/Ecto- | 400 | 1.852 | 9.31 | 5.279 | 285.40% |
| Rotylenchus (spiral) | Endo-/Ecto- | 500 | | | | |
| Paratrichodorus (stubby-root) | Ecto- | 100 | 39 | 116 | 0 | −100.00% |
| Tylencorhynchus (stunt) | Ecto- | 300 | 0 | 0 | 102 | |
| Pratylenchoides | Endo- | 80 | | | | |
| Xiphinema (dagger) | Ecto- | 100 | | | | |
| Belonolaimus (sting) | Ecto- | 10 | | | | |
| Gracilacus (pin) | Ecto- | 300 | | | | |
| Aphelenchoides | Endo-/Ecto- | 300 | | | | |
| Ditylenchus | Endo-/Ecto- | 400 | | | | |
| Nematode Damage Index (NDI) | | | 11.4 | 3.8 | 7.8 | −31.58% |

### Example 3—Ozonated Water and Flavonoids are Effective Against Bacteria

A premier league football (soccer) pitch was treated with ozonated water and/or flavonoids. Turf samples were obtained by obtaining soil profiles (including the root zone) from the pitch and treated as follows:

TABLE 4
samples analysed

| Sample | Comments |
|---|---|
| 1 | Control sample before treatment |
| 2 | Control sample before treatment |
| 3 | Ozone treatment only (2 ppm) |
| 4 | Bioflavonoid treatment only (10 ml/L) |
| 5 | Ozone treatment (2 ppm) + Bioflavonoid |
| | treatment (10 ml/L) |
| 6 | Ozone treatment (10 ppm) only |
| 7 | Ozone treatment (6 ppm) only |
| 8 | Ozone treatment (6 ppm) + Bioflavonoid |
| | treatment (10 ml/L) |
| 9 | Ozone treatment (10 ppm) + Bioflavonoid |
| | treatment (10 ml/L) |

Bacterial counts only were performed on these samples. 10 ml of sample was mixed with filtered water. Bacterial counts were done at between 100 and 300 to 1 dilution. One drop of the dilution was transferred onto a slide and observed under a bright field microscope.

The results of these bacterial counts are shown below:

TABLE 5

| | Bacteria | |
|---|---|---|
| Sample | (microgram per ml) | Notes |
| 1 | 177 | Mainly motile cocci/rods |
| 2 | 149 | Motil cocci present in big number |
| 3 | 121 | Cocci/rods |
| 4 | 117 | |
| 5 | 100 | |
| 6 | 89 | Few motile bacteria round and rod shapes |
| 7 | 86 | Many motile bacteria cocci and rod shape, |
| | | flagellate seen |
| 8 | 72 | Spirochetes present, frequent flagellates, |
| | | motile rods/cocci |
| 9 | 67 | Mainly cocci, saw few sized flagellates |

These results demonstrate that the control samples showed the highest levels of bacteria. The combination of ozone with flavonoid treatment leads to dramatic decreases in bacterial levels as compared to application of ozone or flavonoids alone.

The amount of ozonated water and/or flavonoids that are applied to a crop may also be optimised to maintain the most effective fungi to bacteria biomass ratio for the particular plant species.

Typical fungi to bacterial biomass ratios for a range of commercial species are shown below:
F:B=0.1—weedy stage, or irrigated wheat (not much biomass, or highly bacterial due to use of chemicals). Example plant-crabgrass.
F:B=0.3—early successional plants (early annuals, dryland wheat). Bromus, bermuda, brassicas, mustard and kale crops as examples.
F:B=0.75-0.8—mid successional grasses, vegetables, herbs and forbes
F:B=1-late successional grasses, productive row crops, pastures, turf, prairies (fescues, corn, wheat, luceme)
F:B=2-5—fruit bushes
F:B=5-100—deciduous trees, orchards
F:B=100-1000—late successional, old growth, conifer systems

In every case both bacterial and fungal feeders need to be present in order for nutrient cycling to occur. In general, aerobic conditions promote the development of beneficial elements of the soil food web, and anaerobic conditions promote the development of the opportunistic, detrimental elements. On the flip side, beneficial bacteria and fungi through their activity build the porous structure of compost and soil, which in turn allows for water and oxygen to penetrate as deep as this structure exists.

The compositions of the invention may be applied in an amount effective to achieve a ratio of fungi to bacteria of about 0.5 to about 1.5 preferably about 1:about 1.

### Example 4—Bacterial Counts on Plastic Grass Samples

The effect of ozone on bacteria was determined by experiments performed at the T.I. Soil Ecology Laboratory, UK. 10 ml of artificial grass clippings were added to 40 ml of water. One drop of the dilution was transferred onto a slide and observed under a bright field microscope. Bacterial counts were performed between 5 and 10 to 1 dilutions. One drop of the dilution was transferred onto a slide and observed under a bright field microscope.

The results are presented in Table 6 below:

| Sample | Bacteria microgram per ml |
|---|---|
| Control | 2.7 |
| 2.5 ppm Ozone | 2.5 |
| 5 ppm Ozone | 1.1 |
| 8 ppm Ozone | 0.8 |
| Control - liquid | 5 |
| 2.5 ppm - liquid Ozone | 3 |
| 5 ppm - liquid Ozone | 2 |
| 8 ppm - liquid Ozone | 0 |

The number of bacteria are very low in the “leaf” portion of the turf, but nevertheless there is a visible trend in the reduction of the numbers in proportion to the amount of treatment applied. To ensure this result, the test was carried out twice-once with the method described above, and once with taking a drop out of the fluid itself.

### Example 5—Algae Counts on Plastic Grass Samples

The effect of ozone on algae was determined by independent experiments performed at the T.I. Soil Ecology Laboratory, UK. 10 ml of artificial grass clippings were added to 40 ml of water. One drop of the dilution was transferred onto a slide and observed under a bright field microscope. Algal counts were performed between 5 and 10 to 1 dilutions. One drop of the dilution was transferred onto a slide and observed under a bright field microscope.

The results are presented in Table 7 below:

| Sample | Algae cell per ml |
|---|---|
| Control | 40760 |
| 2.5 ppm Ozone | 20380 |
| 5 ppm Ozone | 0 |
| 8 ppm Ozone | 0 |
| Control - liquid | 89672 |
| 2.5 ppm - liquid Ozone | 73368 |
| 5 ppm - liquid Ozone | 73368 |
| 8 ppm - liquid Ozone | 16304 |

The results represent the number of algal cells per ml of sample. In addition to the method described above, the water was sampled in the bag directly. In both cases, there is a trend correlating the treatment to the reduction in the number of cells observed. It is much more clearly visible in the sampling of the water itself rather than the “leaf” dilution.

### Example 6—Application of Ozone and Nano-Coating onto Artificial Pitch

An application of ozone and nano-coating developed for artificial pitches was tested at Arsenal, London. The dissolved ozone is manufactured on the fly and sprayed directly onto the playing surface.

The nano-coating forms a transparent film with acid modification carrying a positive zeta potential which is responsible for the electrostatic attraction of polar pathogens, micro-organisms and volatile organic compounds towards the surface. The applied dry coating shows the following three modes of action:
(A) Due to the positive surface charge, polar organic compounds (e.g., acetone, formaldehyde) as well as “negatively charged” germs get attracted toward the surface.
(B) Protons from the surface might attack the nitrogen centres from proteins sitting in the cellular outer membrane. Through this protonation, the proteins alter their 3-dimensional angle. The outer membrane becomes perforated and cell fluid can leak out killing the germ.
(C) Silver ions can bind irreversibly to enzymes or the DNA and accelerate the elimination of pathogens.

Pieces of artificial pitch (10×10 cm) and Petri dishes were coated with the nano-coating a water-based emulsion comprising 0.1% titanium dioxide, silver chloride and silicum dioxide) and incubated with various pathogen and microorganisms.

In both test systems, ISO 22196 (high humidity, LED light) and ISO 27447 (ambient humidity, UVA light) the nano-coating demonstrates a strong efficacy against suspensions of E. Coli and S. aureus (see FIG. 5). There is even a pronounced fungicidal efficacy against a highly robust mould/spores like Aspergillus brasiliensis (ISO27447 test) under real outdoor conditions (see FIG. 6).

Field tests were subsequently performed with a preparation of the nano-coating after treating artificial grass with ozonated water (see FIG. 7). The test set up involved the definition of 5 sampling places (1-5) with agar plates (total germ count; TSA Lethen) (FIG. 7A). The first sampling before disinfection was taken at 10:30 to 10:40. The second sampling was taken after disinfection with ozone (11:00 to 11:20), and the third sampling taken after coating with the nano-coating developed for artificial pitches (11:40 to 11:50).

The results (FIGS. 7B and 7C) reveal the central areas #2 to #4 show the highest pathogen and micro-organism load. Continuous pathogen and microorganism reduction was observed after treatment with ozone (6 ppm) and the nanocoating.

In summary, ozonated water is manufactured in the field and sprayed directly onto the playing surface. The subsequent nano-coating is capable of lasting 6 months providing an active cleaning environment on the playing surface.

1. A method of controlling one or more pathogens comprising delivering an effective amount of ozone and one or more pathogen reducing compound(s) by spraying onto a grassed, artificial or hybrid pitch surface.

2. The method of claim 1, wherein the pathogen reducing compound(s) are flavonoids and optionally wherein the flavonoids are sprayed onto a grassed or hybrid pitch surface.

3. (canceled)

4. The method of claim 1, wherein the pathogen reducing compound(s) provide a nano-coating to the pitch surface.

5. The method of claim 4, wherein the surface is an artificial pitch.

6. The method of claim 4, wherein:
(i) the nano-coating has hydrophilic properties and/or is photocatalytic; and/or
(ii) the nano-coating is in the form of nanoparticles having an average particle size of about 10 nm, specific surface area of about 130 m/g and/or grain size of about 42±16 nm.

7. The method of claim 4, wherein the pathogen reducing compound comprises at least one metal oxide and optionally wherein the pathogen reducing compound further comprises an inorganic metal (optionally copper, silver or manganese), non-metal (optionally fluorine or calcium) or two-dimensional material (optionally MXenes, MOF or graphdiyne).

8. (canceled)

9. The method of claim 7, wherein the metal oxide is titanium dioxide and optionally wherein the pathogen reducing compound is a water-based suspension comprising about 0.1% titanium dioxide optionally wherein the suspension further comprises silver chloride and/or silicum dioxide.

10. (canceled)

11. The method of claim 1, wherein the pathogen reducing compound(s) are delivered by spraying after an initial treatment with the ozone.

12. The method of claim 1, wherein the pathogen reducing compound(s) are mixed with ozonated or non-ozonated water using a spray nozzle configured to combine flow from at least two separate streams.

13. The method of claim 1, wherein the ozone and pathogen reducing compound(s) are delivered using a retractable spray lance.

14. The method of claim 1, wherein the ozone and/or pathogen reducing compound(s) are delivered at a rate between about 0.001 ppm to about 50 ppm.

15. The method of claim 1, further comprising delivering one or more oxidizing reagents, optionally wherein the oxidizing reagents are oxygen (O₂) and/or hydrogen peroxide.

16. The method of claim 1, wherein the pathogens are fungi, insect, nematode, oomycete, bacteria, virus, viroid, virus-like organisms, phytoplasma, protozoa and/or parasitic plants, and optionally wherein the pathogen is a parasitic nematode.

17. (canceled)

18. The method of claim 1, wherein the ozone is prepared on-site by electrolysis prior to delivery.

19. The method of claim 1, wherein the ozone and pathogen reducing compound(s) are applied to the pitch surface one or more times.

20. The method of claim 1, wherein the method further comprises determining the number of pathogens in a sample obtained from the site of infection after treatment with the ozone and/or pathogen reducing compound(s).

21. The method of claim 20, wherein, if the number of pathogens after treatment are above a threshold level, a second amount of ozone and/or pathogen reducing compounds are applied to the site of infection.

22. The method of claim 20, wherein the number of nematodes is determined, and the threshold level is a nematode damage index (NDI) of about 10.

23. The method of claim 20, wherein the number of bacteria and/or fungi is determined.

24. The method of claim 1, wherein the ozone and one or more pathogen reducing compound(s) are applied in an amount effective to achieve a ratio of fungi to bacteria of about 0.5 to about 1.5.
